(19) United States
(12) Patent Application Publication
(10) Pub. No.: US 2018/0249911 A1
(43) Pub. Date: Sep. 6, 2018

(54) DIFFUSING WAVE SPECTROSCOPY APPARATUS AND CONTROL METHOD THEREFOR

(71) Applicant: Canon USA Inc., Melville, NY (US)

(72) Inventors: Masaki Hosoda, Cambridge, MA (US); Haruo Nakaji, Boston, MA (US)

(21) Appl. No.: 15/449,195

(22) Filed: Mar. 3, 2017

(51) Int. Cl. A61B 5/00; G01D 5/347; G01N 21/47

(52) U.S. Cl. CPC A61B 5/0075; G01D 5/347; G01N 2201/127; G01N 2021/4742; G01N 21/474

(57) ABSTRACT

A DWS apparatus includes a coherent light source, a photodetector, a control unit which can measure an intensity autocorrelation function, a measuring unit which can measure a source-detector distance to obtain source-detector distance data, and a calibrating unit which adjusts the intensity autocorrelation function by using the source-detector distance data. The calibrating unit calibrates the intensity autocorrelation function by adjusting the time constant of the autocorrelation function based on a comparison of the source-detector distance to the time constant of the intensity autocorrelation function.

### BACKGROUND

### Field

The disclosure of this patent application relates generally to optical imaging, and in particular it relates to a diffusing wave spectroscopy apparatus including an encoder that measures source-detector distance, and control methods therefor.

### Related Art

Diffusing wave spectroscopy (DWS), also known as diffuse correlation spectroscopy (DCS), is a useful technology to detect particle motion in turbid media using coherent light. In DWS, coherent light irradiates a sample (e.g., blood or tissue), the light scattered by the particles in the sample is collected and guided to a detector, and constructive and destructive interference is observed as a random granular pattern of spots (speckle). When scattering particles move in the sample, speckle intensity detected by the detector fluctuates. By counting photon series caused by speckle intensity fluctuation, particle movement in a sample can be estimated precisely. In the case of blood or tissue, the primary moving particles (scatterers) are red blood cells (RBCs).

Recently, DWS has been actively investigated as a valuable tool for non-invasively examining various properties of biological tissue. In particular, DWS has become a tool of choice for analyzing blood flow characteristics (hemodynamics) in biological tissue, for diagnosis of disease, and for continuous monitoring and evaluation of therapeutic effects in pre-clinical and clinical investigations. For example, the use of DWS technology for assessing blood flow in tissue of a patient is discussed in publication US 2012/0184831 A1. The structure of DWS instruments is well known, as described in, for example, U.S. Pat. Nos. 6,076,010, 6,831,741, and 8,463,346.

Although various arrangements are known, the main components in a conventional DWS system are a coherent light source for irradiating light onto a sample, an optical detector for collecting the scattered light, and a controller unit including a correlator for calculating an intensity autocorrelation function which is related to the properties of the sample. FIG. 1 shows a schematic of a typical DWS apparatus 100. Conventionally, a DWS apparatus 100 includes a computer to with a display 20, a light control module 30 containing a light source (LS) 32 and a photodetector (PD) 34, and a probe 50. The computer 10 is connected to the light control module 30 via known electronic circuitry or a networked connection 25, and the light control module 30 is connected to the probe 50 via a cable/fiber bundle 40.

In FIG. 1, coherent light from the light source 32 is guided by an optical fiber of fiber bundle 40 to the probe 50 to irradiate a sample 60 via a source unit (source 52). Scattered light that has diffused through the sample is collected by a detector unit (detector 54) at a distance ρ away from the source 52. The collected light is guided from detector 54 to the photodetector 34 via an optical fiber of the fiber bundle 40. The photodetector 34 generates an electrical signal corresponding to the intensity of the light collected by the detector 54. A computer 10 calculates an autocorrelation function g2(τ) from measurements of photon intensity. The optical properties of the sample (e.g., blood flow) is estimated by fitting the measured autocorrelation function to mathematical models appropriate to the type of measurement being performed.

In DWS, the source-detector distance ρ (rho) is an important parameter that affects the time constant value of the autocorrelation function g2(τ). Since the time constant value is one of the most valuable parameters of information extracted from the g2(τ) function, it is important to ensure that this parameter is appropriately measured. In the related art of U.S. Pat. No. 6,831,741 various source-detector distances are contemplated for deriving properties of a sample; U.S. Pat. No. 8,463,346 describes devices where a greater source-detector spacing allows light to reach deeper into the tissue volume; patent application publications US 20140206980 and US 20150276571 disclose DCS instruments showing the dependence on source-detector separation for detection depth and blood-flow rate sensitivity. Therefore, it is evident that source-detector distance is an important parameter in the calculation of the autocorrelation function g2(τ).

However, when a patient moves and the actual (physical) source-detector distance is changed, the time constant τ is varied. In addition, the probes used for DWS measurements are rigid or semi-rigid probes which make it difficult to accurately apply the probe to a patient's surface because of the differences in anatomy and location (e.g., head, leg, arm or finger) of different patients (e.g., neonates vs. adults) do not conform to standardized probes. Specifically, while rigid flat probes are useful in certain anatomies and locations of certain patients, such rigid flat probes may not accurately conform to non-flat surfaces of other anatomies of patients. On the other hand, while semi-flexible probes can be forced to conform to the head or other parts of a patient's anatomy, these probes may not remain stable for long-term measurements. In highly unstable or delicate patients, the pressure required to deform a semi-flexible probe or to secure a rigid flat probe onto a patient may be unsafe. More importantly, the flexibility of these semi-rigid probes is usually at the expense of changes in source-detector separation, which causes increased uncertainty on the measured hemodynamic values. For example, as described in publication US 20160361017 a change in source-detector separation of only about 0.5 mm can result in changes in detected signal and calculated physiological properties.

The time constant τ is also varied by changing the blood flow dynamics (e.g., blood cell concentration, blood pressure, flow resistance due to plaque, and so on). As a result, it is difficult to identify the reason why the time constant τ is changed, and this could result in inaccurate diagnosis of a patient and/or erroneous measurement of the properties of a sample.

### SUMMARY

An object of the present patent application is to improve DWS measurement results by adding a mechanism to measure the distance between the source and detector in the probe to obtain accurate source-detector distance information. The source-detector distance information can then be used in a feedback loop to calibrate time decay variations of the correlation function g2(τ) caused by patient movement, subject anatomy or probe accommodation.

More specifically, although blood flow dynamics such as blood cell concentration, blood pressure, flow resistance, etc., cannot be easily controlled because these parameters would generally depend on the specific physical conditions of a sample (subject), an accurate measurement of the actual source-detector distance parameter needs to be improved in order to provide correct diagnosis even if a subject under examination moves or a measurement is performed on patients having tissue with different physical conditions or different anatomies.

According to at least one embodiment of the present application, a DWS apparatus includes a coherent light source, a photodetector, a control unit which can measure an intensity autocorrelation function, a measuring unit which can measure a source-detector distance to obtain source-detector distance data, and a calibrating unit which adjusts the intensity autocorrelation function by using the source-detector distance data.

Further features and advantageous of the invention will become apparent to those skilled in the art from the following description of exemplary embodiments with reference to the attached drawings.

### BRIEF DESCRIPTION OF DRAWINGS

FIG. 1 illustrates a schematic of a conventional diffusing wave spectroscopy (DWS) system.

FIG. 2A illustrates a schematic of a diffusing wave spectroscopy (DWS) apparatus, according to an embodiment of the present patent application. FIG. 2B illustrates an exemplary DWS probe in the form of a clamp.

FIG. 3A is a graphical plot illustrating a relation dependence of the time constant (T) on the variation of source-detector distance p. FIG. 3B is graph to explain principles of source-detector distance measurement using a rotary encoder.

FIGS. 4A and 4B show an example of a DWS probe in the form of a clamp including a rotary encoder to measure the source-detector distance.

FIGS. 5A1-5A2 show an example of a DWS probe in the form of a clamp including one linear encoder to measure the source-detector distance. FIGS. 5B1-5B2 show an example of a DWS probe in the form of a clamp including multiple linear encoders to measure the source-detector distance.

FIG. 6 illustrates a schematic block diagram of a control system for performing DWS measurements and calibrating the autocorrelation function by using source-detector distance information.

FIG. 7 illustrates a flow diagram of a first process for calibrating the time constant (τ) using source-detector distance data.

FIG. 8 illustrates a flow diagram of a second process for calibrating the time constant (τ) using source-detector distance data.

FIG. 9 is a diagram to explain how the algorithm of FIG. 8 makes use of actual source-detector distance to adjust the autocorrelation function.

FIG. 10 shows an example of an autocorrelation function g2(τ) with a plateau level c.

### DETAILED DESCRIPTION

In the following description, reference is made to the accompanying drawings which are illustrations of embodiments in which the disclosed invention may be implemented and practiced. It is to be understood, however, that those skilled in the art may develop other structural and functional modifications without departing from the novelty and scope of the instant disclosure.

In referring to the description, specific details are set forth in order to provide a thorough understanding of the examples disclosed. In other instances, well-known methods, procedures, components and circuits have not been described in detail as not to unnecessarily lengthen the present disclosure. Some embodiments of the present invention may be practiced on a computer system that includes, in general, one or a plurality of processors for processing information and instructions, random access (volatile) memory (RAM) for storing information and instructions, read-only (non-volatile) memory (ROM) for storing static information and instructions, a data storage devices such as a magnetic or optical disk and disk drive for storing information and instructions, an optional user output device such as a display device (e.g., a monitor) for displaying information to a user, an optional user input device including alphanumeric and function keys (e.g., a keyboard) for communicating information and command selections to the processor, and an optional user input device such as a pointing device (e.g., a mouse) for communicating user input information and command selections to the processor.

As will be appreciated by those skilled in the art, the present examples may be embodied as a system, method or computer program product. Accordingly, some examples may take the form of an entirely hardware embodiment, an entirely software embodiment (including firmware, resident software, micro-code, etc.) or an embodiment combining software and hardware aspects that may all generally be referred herein as a “circuit”, “module” or “system”. Further, some embodiments may take the form of a computer program product embodied in any non-transitory tangible medium of expression having computer-usable program code stored therein. For example, some embodiments described below with reference to flowchart illustrations and/or block diagrams of methods, apparatus (systems) and computer program products can be implemented by computer program instructions. The computer program instructions may be stored in computer-readable media that when executed by a computer or other programmable data processing apparatus causes the computer or processing apparatus to function in a particular manner, such that the instructions stored in the computer-readable media constitute an article of manufacture including instructions and processes which implement the function/act/step specified in the flowchart and/or block diagram.

The terms first, second, third, etc. may be used herein to describe various elements, components, regions, parts and/or sections. It should be understood that these elements, components, regions, parts and/or sections are not limited by these terms of designation.

These terms of designation have been used only to distinguish one element, component, region, part, or section from another region, part, or section. Thus, a first element, component, region, part, or section discussed below could be termed a second element, component, region, part, or section merely for purposes of distinction but without departing from structural or functional meaning.

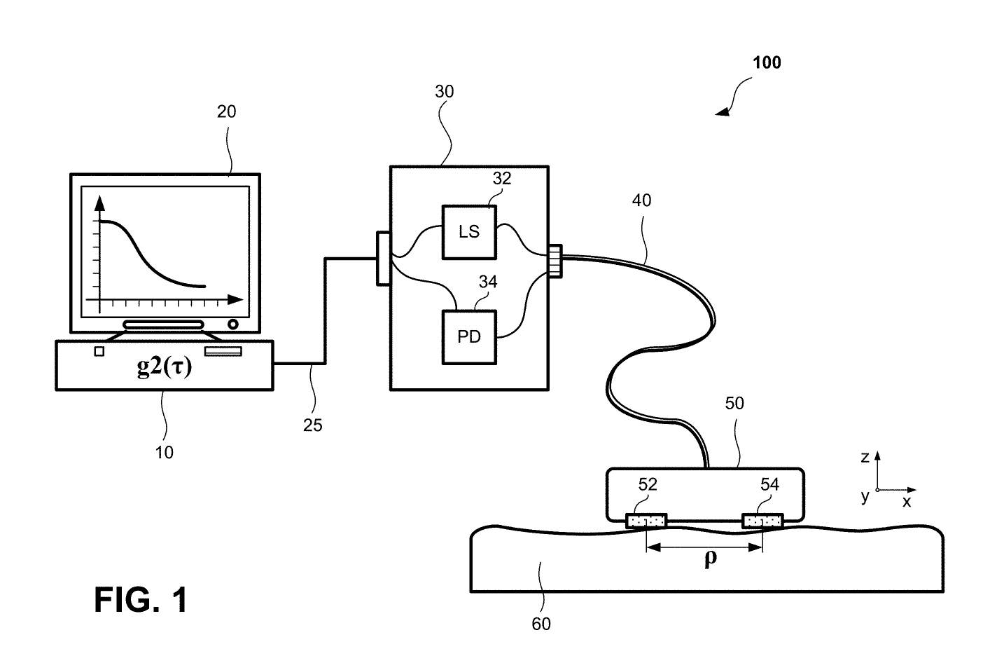

Exemplary embodiments are described below in more detail with reference to the several drawings where like reference numerals refer to like parts. FIG. 1 is/illustrates a block diagram of a conventional Diffusing Wave Spectroscopy (DWS) apparatus 100.

### <DWS Principles and Need for Accurate Source-Detector Distance Calculation>

In general, when light hits small particles such as blood cells within tissue, as long as the particles are small compared to the wavelength of light, the light scatters in all directions (Rayleigh scattering). Even if the light source is a laser, and thus light is monochromatic and coherent, the scattering intensity fluctuates over time. This fluctuation is caused because small molecules in liquid solutions undergo Brownian motion, and so the distance between the scatterers in the solution is constantly changing with time. The scattered light then undergoes either constructive or destructive interference by the surrounding particles, and within this intensity fluctuation, information is contained about the time scale of movement of the scatterers. The dynamic information of the particles is derived from an autocorrelation of the intensity trace recorded during DWS measurements.

As it is known to persons having ordinary skill in the art, the normalized (second-order) autocorrelation function g2(τ) for a transmission condition of scattered light through particles in turbid media is defined by Equation (1) using what is known as the “Siegert” relation. Equation (1) relates the second-order autocorrelation function with the first-order autocorrelation function g1(τ) as follows:

g₂(τ)=1+β|g₁(τ)|² Equation (1)

where

$${g}_{1} ( τ ) = {\int }_{0}^{∞} P ( s ) {e}^{- ( 2 τ / t \frac{s}{{l}^{*}} )} ds ,$$

Equation (1a), represents correlation for a monodisperse sample modeled as an infinite slab.

In Equation (1), β (beta) is a coherence factor; τ (tau) is the time lag. In Equation (1a), P is the probability or fraction of scattered intensity (fraction of photons) which travels a path length s through the sample (scattering medium); that is, s in Equation (1a) is the path length of a single photon passing through the sample. And t=1/k₀²D, where k₀ is a wave number of the light used to irradiate the sample, D is the particle diffusion coefficient, and l* is a mean free path length.

The mean free path length l* is the averaged distance between randomized scattering events in a suspension with very small particles. The mean free path length l* depends primarily on the number of target particles per unit volume, and on the effective cross sectional area for collision. The photon path length s is the total length of photon trajectory with N times scattering events in the suspension. Then a change of a source-detector distance does not affect the mean free path length l*, but it does affect the photon path length s.

Since the relationship between the source-detector distance ρ and the time constant τ can be used to characterize the scattering medium, the inventors herein have determined that it is advantageous to accurately measure the source-detector distance, and to use the source-detector distance data to calibrate the autocorrelation function g2(τ). This is considered to be particularly useful in cases where source-detector distance is intentionally or unintentionally changed during examination.

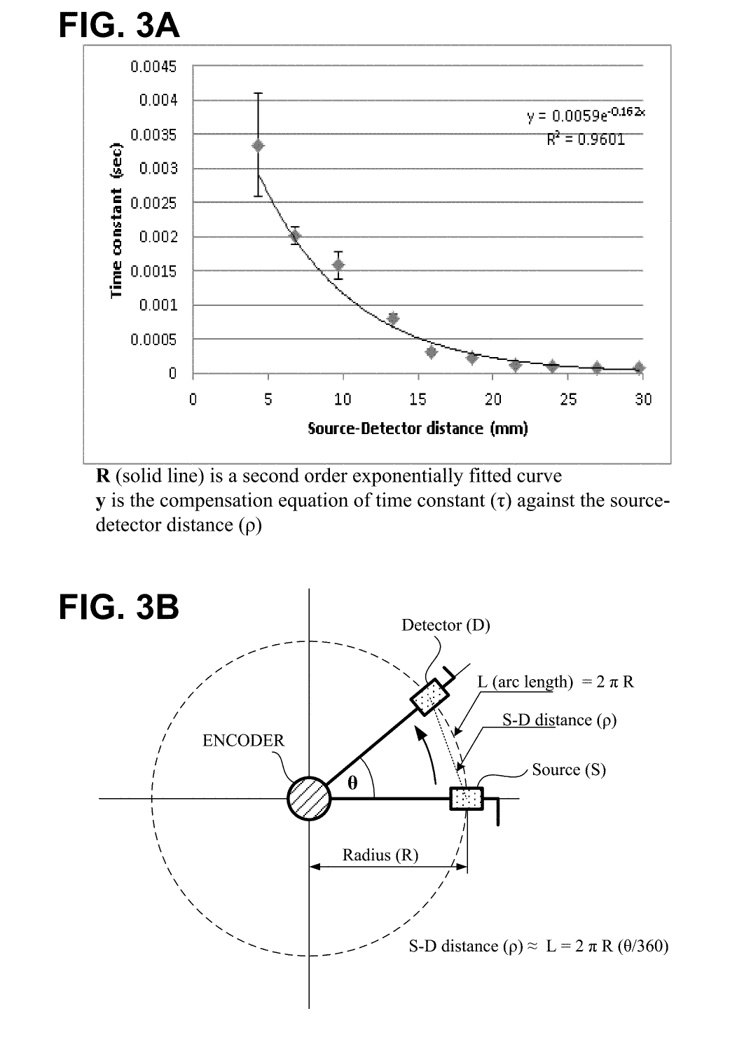

FIG. 3A is a graph which shows results of an exemplary experiment performed by the inventors herein to demonstrate how the source-detector distance affects the time constant values calculated from intensity autocorrelation functions, g2(τ).

In FIG. 3A, the horizontal axis represents the source-detector distance in the unit of millimeters (mm) and the vertical axis represents the time constant (i) values with their standard deviations in the unit of seconds (sec). The time constant values are calculated by using an exponential fitting of the autocorrelation function g2(τ). The solid line in FIG. 3A is an exponentially fitted curve with R²=0.96. The sample for this experiment was 0.25% concentration Intralipid®. The source light was delivered from a laser diode through a single mode fiber (SMF) to the sample, and the scattered light was collected by a different SMF that was connected to a photodetector. The source SMF and the detector SMF were set diagonally from each other, and the SMF tip ends (distal ends) were in contact (touching) with the sample. In this experiment preformed by the inventors, numerous DWS measurements were performed with source-detector distances varying in small incremental steps from approximately 5 mm to 30 mm, as shown on the horizontal axis of FIG. 3A.

As discussed above, the source-detector distance affects the time constant value, which is one of the most valuable information parameters extracted from the correlation function g2(τ). Therefore, it is important to obtain the actual (physical) source-detector distance and calibrate the time constant value using the source-detector distance information. In order to provide an affordable and easy to implement solution to the source-detector distance measurement the inventors herein have proposed adding to the DWS probe a source-detector distance measurement unit in the form of an encoder mechanism.

### First Embodiment

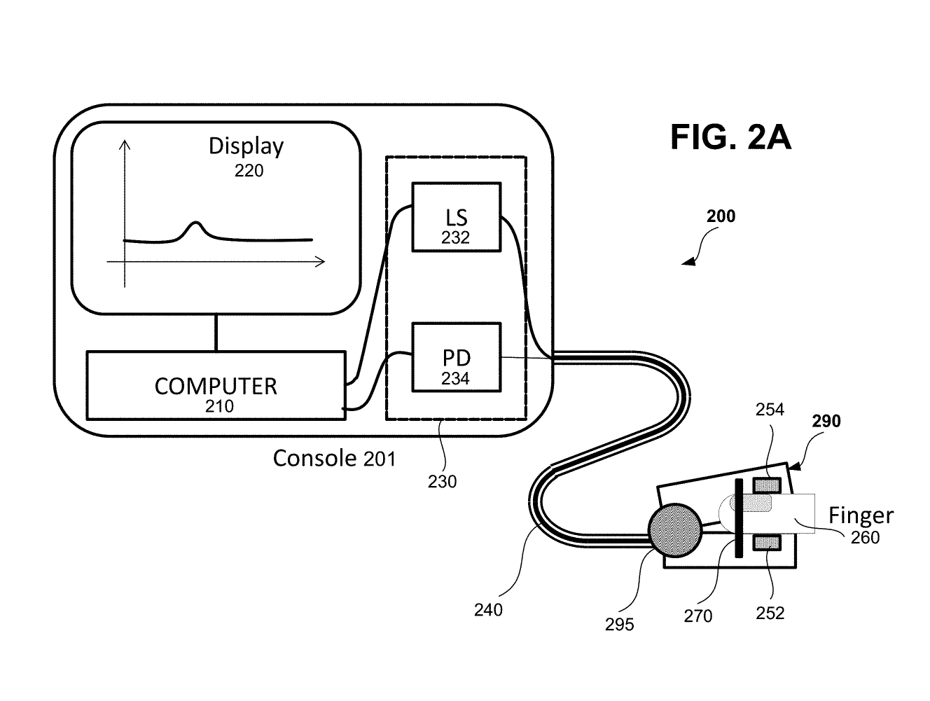

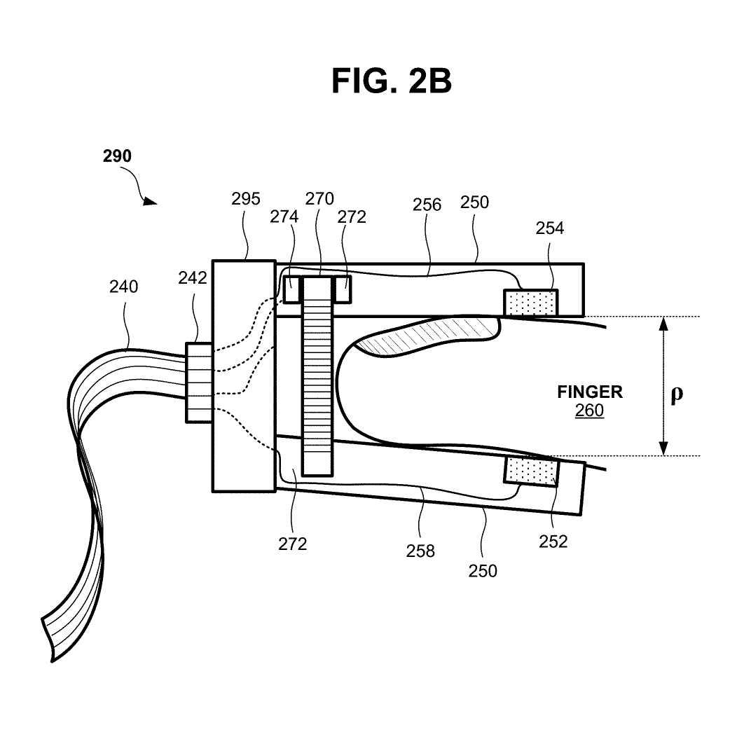

FIG. 2A illustrates a diffusing wave spectroscopy (DWS) apparatus according to one embodiment of the present patent application. In FIG. 2A, a DWS apparatus 200 includes a DWS probe 290 connected to an operating console 201 via a cable/fiber bundle 240. The operating console 201 is constituted of known DWS controlling parts including a computer 210, a display 220 and a light control module 230. The light control module 230 includes a laser light source (LS) 232 and a photodiode (PD) (photodetector 234) similar to the above-describe light source 32 and photodetector 34 of FIG. 1. The DWS probe 290 is illustrated as a finger clamp composed of a pair of clamp plates joined by a mechanical hinge portion 295 and configured to receive therein a finger 260 (an anatomical extremity). The DWS probe 290 includes a source 252, a detector 254, and an optical encoder 270 which serves to obtain source-detector distance, as described below more in detail.

FIG. 2B illustrates a configuration of a DWS probe 290 including a source-detector measurement unit. In FIG. 2B, the DWS probe 290 is illustrated as finger clamp configured to receive therein an anatomical extremity, such as a finger or a toe. However, the principles and structure of the source-detector measuring unit disclosed herein can be applied to any type of DWS probe in which a source and detector exist. In the illustration of FIG. 2B, the probe 290 includes an encoder 270, a hinge portion 295, and a pair of clamp plates 250 which are mechanically joined by the hinge portion 295. Since the clamp plates 250 are mechanically joined by the hinge portion 295, the clamp plates 250 can be opened and closed to allow a finger 260 to be accommodated therebetween.

To perform DWS measurements, a source terminal (source 252) is arranged on one of the clamp plates 250, and a detector terminal (detector 254) is arranged on the other one of the clamp plates 250. In FIG. 2B, the source 252 and the detector 254 are shown as being positioned substantially at the proximal end of the clamp plates 250. However, the arrangement of the source 252 with respect to the detector 254 is not limited to any specific geometry or location, as long as both the source 252 and detector 254 are located at a predetermined distance (ρ) from each other, and both are in contact with the tissue of finger 260 (in contact with the sample).

The encoder 270 is an example of source-detector distance measurement unit. As it is known to persons having ordinary skill in the art, encoders provide a measurement of the position of a component in a system relative to some predetermined reference point. As used herein, the term “encoder” is meant to describe any mechanical, electronic, optical, magnetic and/or ultrasonic device, including combinations thereof, where such device can be used to obtain source-detector distance information in a DWS probe, as fully explained below.

Encoders are typically used to provide a closed-loop feedback to a system. Those skilled in the art will appreciate that there are numerous types of encoders, and this patent application does not intend to describe all types of encoders. For purposes of illustration and example, optical encoders are described herein, but the principles of using an optical encoder in a DWS probe can be applied to other types of encoders as well. Optical encoders can be linear or rotary and transmissive or reflective. A rotary encoder outputs a digital signal that indicates the position of a rotating element relative to some known reference position that is not moving. A rotary encoder can provide a measurement of either the absolute angle of rotation or incremental changes in the angle of rotation or a rotary shaft or a component attached to the rotary shaft. Some rotary encoders may also provide an indication of the direction of rotation (i.e., direction of movement). A linear encoder measures the distance between a present position of a linear moveable element (stripe) and a reference position that is fixed with respect to the moveable element as it moves along a predetermined path. Optical encoders therefore utilize a light source such as an LED and a photo detector such as a photodiode to measure changes in the position of an encoded disk or stripe.

In a transmissive rotary encoder, an encoded disk includes a series of alternating opaque and transparent strips. The light source is located on one side of the disk, and the photodetector is located on the other side. The light source and photodetector are fixed relative to one another, and the code strips move therebetween such that the light reaching the photodetector is interrupted by opaque regions of the disk. The position of the encoding disk is determined by measuring the transitions between the light and dark regions observed by the photodiode. The same applies to a transmissive linear encoder with the difference that a linear carriage encoded with a series of alternating opaque and transparent strips moves in a linear path between light source and photodetector fixed relative to one another.

In a reflective encoder, the light source and photodetector are located on the same side of the encoding strip, and the encoding strip consists of alternating reflective and absorbing stripes. The light source is positioned such that light from the light source is imaged into the detector when the light is reflected from the reflective strips.

Whether it is a linear or rotary, transmissive or reflective encoder, when the coded element moves with respect to the light source and the detector, the light beam is intermittently interrupted by the opaque stripes of the coded element, and therefore photodiodes in the detector receive intermittent flashes of light. The resultant signal is then used to generate a logic signal that transitions between logical one and logical zero. This logic signal is digitized/quantized and used in a feedback loop. It should be noted that the foregoing basic description of optical encoders is provided to inform the reader of the general principles of conventional encoders. Persons of ordinary skill in the art will appreciate that several other types of optical and non-optical encoders exist which function substantially in the general manner described above. It will be therefore a matter of design choice to choose the type of encoder and encoding scheme to obtain the most accurate source-detector distance for the various DWS probes disclosed herein.

Turing back to FIG. 2B, the encoder 270 is preferably a conventional compact and self-contained optical encoder that includes its own light source 272 (encoder light source) and detector 274 (encoder detector). The encoder 270 includes non-shown interface terminals (electrical connections) to be connected to a cable/fiber bundle 240 via a connector 242. In the probe 290, the source 252 receives light from the light source 232 (shown in FIG. 2A) via an optical fiber 258 which is connected to the cable/fiber bundle 240 via the connector 242. Similarly, the detector 254 of the probe 290 transmits collected light to the photodetector 234 (shown in FIG. 2A) via an optical fiber 256 which is also connected to the cable/fiber bundle 240 via the connector 242. The cable/fiber bundle 240 is connected to the light control module 230, as illustrated in FIG. 2A.

In the DWS apparatus 200 of FIG. 2A, the coherent laser light emitted from the laser source (LS) 232 is coupled with an optical fiber (preferably a single mode fiber), which then carries the laser light to finger 260 (a sample). In the embodiment shown in FIG. 2A, the light source 232 can be a diode pumped solid state (DPSS) laser, a volume holographic grating laser diode (VHG LD), a vertical cavity surface emitting laser (VCSEL), or the like.

The source terminal (source 252) in FIGS. 2A-2B denotes either of a fiber end (a distal end or tip of a fiber), a waveguide, or conventional optics, such as a prism or a lens group configured to illuminate coherent light received from the light source 232 via the optical fiber 258 onto an area (point) of the sample (finger 260 in FIG. 2B). Here, it should be understood that source 252 can also denote an actual light source. More specifically, a VCSEL light source can be placed directly on the probe itself, so that the light is delivered onto the sample more accurately and efficiently. The light penetrates into the sample, is scattered in the sample, and at least part of the scattered light and diffused through the sample is collected by the detector terminal (detector 254). The detector 254 can also be implemented as a fiber end, a waveguide, or conventional optics, such as a prism or a lens group, or combinations thereof. However, similar to source 252, the detector 254 can also be implemented as an actual photodiode or any other photon counting device integrated directly within the probe 290. That is, the detector 254 can also denote a photodetector.

The light collected by the detector 254 is carried to the photodetector 234 via an optical fiber 256. The photodetector 234 may be implemented as an avalanche photodiode (APD), an array of APDs, or a similar photon counting photodetector. The signal output from photodetector 234 is transferred to a control unit (computer 210) for signal and data processing. The photodetector 234 can be integrated with an analog-to-digital (A/D) circuit that generates electrical Transistor-Transistor Logic (TTL) digital pulses, so that the signal output from photodetector 234 can be transferred to the computer 210 as a digital signal composed of TTL pulses. Alternatively, the electrical signal output from photodetector 234 is transferred to computer 210 in analog format, and known circuitry in the computer 210 digitizes the electrical signal for processing therein.

The computer 210 uses a distribution of arrival times of the TTL pulses to quantify temporal fluctuations of the detected light intensity. In this manner, the computer 210 calculates an intensity autocorrelation function g2(τ) from the signal which is output from the PD and related to detected optical intensity. Normally, the intensity autocorrelation function, g2(τ), can be calculated by a dedicated hardware correlator, but due to the limited speed of hardware correlators and the advance in computing power of new computers, it is now possible to compute the autocorrelation function using software correlators. See, for example, Wang et al., “Fast blood flow monitoring in deep tissues with real-time software correlators”, BIOMEDICAL OPTICS EXPRESS, Vol. 7, No. 3, 1 Mar. 2016, which is incorporated by reference herein in its entirety. Therefore, the autocorrelation function can be calculated by any of a hardware correlator, a software correlator module, or a combination of both.

For example, in terms of software processing, according to Wiener-Khinchin theorem, the autocorrelation function g2(τ) can be calculated by using Fourier transform processing algorithms executed by computer 210. And the computer 210 also extracts an experimental parameter β value and a time constant (τ) value from the intensity autocorrelation function. The display 220 can show the g2(τ) function, related parameters such as β values and T values extracted from the g2(τ) function, and the characterization of the sample (e.g., blood flow dynamics). As it is well known to those skilled in the art, examples of a display 220 include a flat panel screen that uses LCE (liquid crystal display) technology, an OLED (organic light emitting diodes) screen rigid or flexible, a projector, or the like.

Referring again to FIG. 2B, the probe 290 (clamping unit) incorporates, in addition to the source 252 and detector 254, the encoder 270 which is an optical rotary encoder (RE) or a linear encoder (LE). Non-optical encoders can also be incorporated into the probe 290 of FIG. 2B or all other probes described herein.

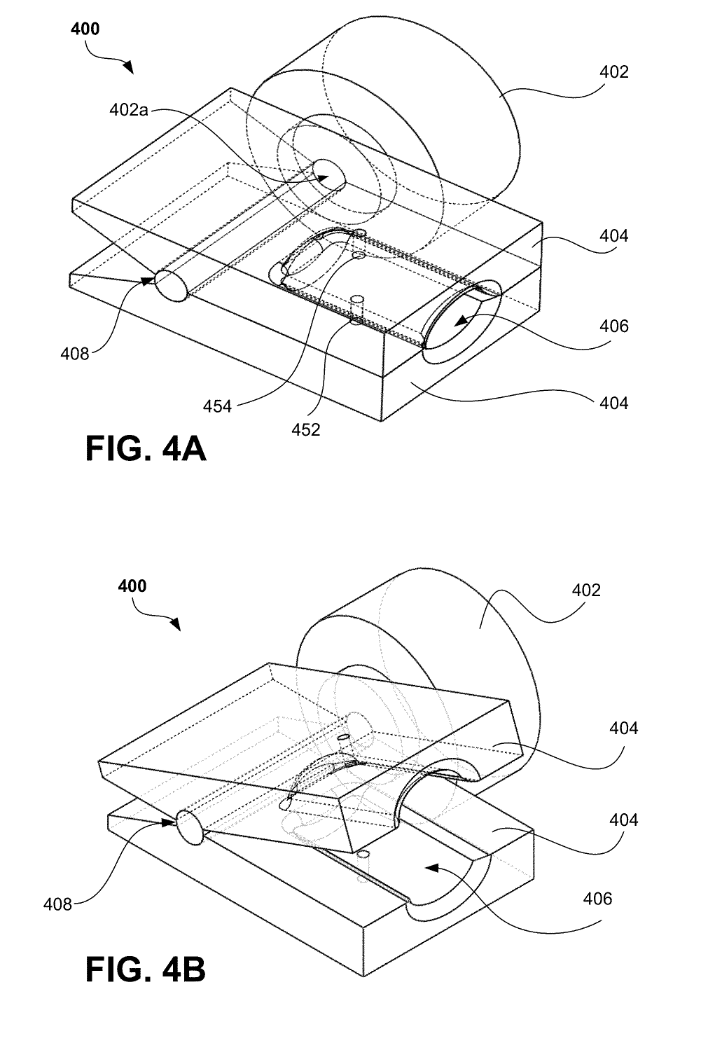

FIGS. 4A and 4B show an example of how a rotary encoder can be integrated into a DWS probe 400. FIG. 4A shows a probe 400 in the form of a finger clamp (clamping unit) in a closed state. FIG. 4B shows the probe 400 in the form of a finger clamp (clamping unit) in an open state. In FIGS. 4A and 4B, the probe 400 shows a rotary encoder 402 incorporated within a hinge portion 408 of the finger clamp. The probe 400 is similar to the previously described probe 290 in that the probe 400 includes a pair of clamp plates 404 that are mechanically joined by the hinge portion 408. The clamp plates 404 each include a notched section to form a sample receiving space 406, which has a tubular shape with a substantially circular cross-section. In this manner, the clamp plates 404 are configured to be opened and closed to receive therein an anatomical extremity of a subject (finger 260) for examination. In the case shown in FIGS. 4A and 4B, a shaft 402a of the rotary encoder 402 is mechanically engaged with the hinge portion 408 of the finger clamp (probe 400), so that an encoded disc of encoder 402 can rotate every time that either one the clamp plates 404 is open and closed, and every time the sample (finger 260) moves or changes its position. FIG. 4A also illustrates the positions where a DWS source terminal (source 452) and DWS detector terminal (detector 454) are respectively located diagonal from each other to deliver light to irradiate the sample and to collect light diffused through the sample.

In the case of using a rotary encoder (RE), as illustrated in FIGS. 4A and 4B, when the RE has a typical resolution of 1000 pulses per revolution (ppr) and the distance between the encoder and the source or between the encoder and the detector is 40 mm, a distance resolution of 0.25 mm can be achieved.

Specifically, when using a rotary encoder in a finger clamp probe 400, as illustrated in FIGS. 4A and 4B, the source-detector distance can be calculated by assuming a separation between source and detector along a circumferential locus (arc) formed when either one the clamp plates 404 is open and closed, and the source-detector distance changes when the sample (finger 260) is placed within the sample receiving space 406. That is, when the probe 400 goes from a closed stated (FIG. 4A) to an open state (FIG. 4B), so that a sample can be inserted into sample receiving space 406, the source-detector distance changes by forming an arc, as shown in FIG. 3B.

As it is known to persons having ordinary skill in the art, the arc length (L) of a circle is given by the formula L=2πR. Therefore, as illustrated in FIG. 3B, the actual source-detector distance (ρ) is given as S-D distance z L=7πR(θ/360). Accordingly, the “distance resolution” in this measurement is the measurement resolution of distance between the source (S) and the detector (D). Here, to explain how the resolution of the rotary encoder affects the measurement resolution of the distance between the source and the detector, an example of a basic rotary encoder is used. A typical resolution for a rotary encoder is 1,000 pulses per revolution (ppr). This means that this rotary encoder produces 1,000 pulses when it rotates 360 degrees (2π radians). Then, the resolution of 1 revolution can be converted to the resolution of distance per pulse using the basic arc length formula as follows:

$$\begin{matrix}resolution of distance = \frac{Arc length}{resolution of revolution} \\ = \frac{2 \times 40 mm \times π}{1 , 000 ppr} \\ = 0.25 mm\end{matrix}$$

Therefore, based on the data of FIG. 3A, a compensation equation of the time constant (τ) as a function of the source-detector distance can be defined as expressed by Equation (2) as follows:

y=0.0059ê(−0.162x) Equation (2),

where y is the value of the time constant (τ) and x is the source-detector distance (ρ).

Then, when the distance resolution for the encoder is 0.25 mm, and assuming a measurement of a finger with 10 mm thickness, the resolution of the time constant (τ) is 4.6×10⁻⁵, which is equivalent to about 4% compared to the original (calculated) time constant, and it is considered to be small enough for purposes of certain measurements.

More specifically, here the “original (calculated) time constant” refers to the calculated time decay (τ) value for a 10 mm finger, which is calculated using the fitted curve R of FIG. 3A as follows:

$$\begin{matrix}{τ}_{10 mm} = 0.0059 {e}^{- 0.162 ρ} \\ = 0.0059 \times {e}^{- 0.162 \times 10 mm} \\ = 1.168 \times {10}^{- 3}\end{matrix}$$

That is, the calculated time decay (τ) value for a 10 mm think finger is 1.168E-3. However, this calculated time decay value can be affected by various factors, such as sample movement, sample misplacement, and including encoder resolution.

Here, to explain how the resolution of the rotary encoder affects the resolution of time constant calculation, the measured source-detector distance is compared to the “original (calculated) time constant” value. Specifically, since the resolution of S-D distance measurement is limited by the rotary encoder resolution of 0.25 mm (in the above example), the actual S-D distance can vary from 10 mm to 10.25 mm, even though the exact measured S-D distance using a rotary encoder should be 10 mm. Then, in the worst case scenario with a rotary encoder having a distance resolution of 0.25 mm, the actual S-D distance can be measured as 10.25 mm. In this case, the time constant then becomes the following.

$$\begin{matrix}{τ}_{10.25 mm} = 0.0059 {e}^{- 0.162 ρ} \\ = 0.0059 \times {e}^{- 0.162 \times 10.25 mm} \\ = 1.121 \times {10}^{- 3}\end{matrix}$$

Then the difference between the S-D distance for the “original (calculated) time constant” value and the actual S-D distance is 1.168E-3-1.121E-3=0.047E-3, as follows:

τ_{10 mm}−τ_{10.25 mm}=4.6×10⁻⁵

The ratio of this difference between the measured S-D distance and the actual S-D distance is approximately 4% as follows:

$$\frac{{τ}_{10 mm} - {τ}_{10.25 mm}}{{τ}_{10 mm}} = 4 \%$$

This means that a typical rotary encoder can measure the actual source-detector distance with at least a 96% accuracy. Generally speaking, a measurement resolution of about 10% is considered acceptable for a general purpose measurement. However, in the case of monitoring the source-detector distance with a rotary encoder, as disclosed herein, it is possible to provide a measurement resolution to within 4% or better from the expected value.

Naturally, when the rotary encoder has higher resolution than a typical resolution, e.g., when using a rotary encoder with 10,000 ppr or when using dual encoders in the probe, the actual source-detector distance can be measured with higher accuracy, and the resolution or accuracy of the time constant (τ) can be further improved.

In the above discussion of encoder distance resolution, the actual S-D distance is considered as the distance defined by an arc length L in order to facilitate the explanation of the distance resolution and its effects on the actual S-D distance. However, as shown in FIG. 3B, the actual S-D distance (ρ) is defined as a linear length between the source (S) and detector (D) terminals, and not necessarily as an are length. However, since the difference between the linear and arc length is very small for the geometries of small anatomical extremities, such as a 10 mm finger or toe, or an even thinner earlobe, the S-D distance can be accurately defined as a linear or are length. For example, when the encoder resolution is 1000 ppr and the probe (clamp) is used to measure a 10 mm thickness extremity, the resolution of distance along the arc length is 0.251327412 mm, and the resolution of distance along the linear length is 0.251326999 mm by the Pythagorean Theorem. The difference is 0.413 μm, which can be considered negligible.

Referring back to Equation (2), once the y time constant value as a function of the actual source-detector distance x is determined, calibration of the correlation function can be performed as theoretically using Equation 1a for every measurement, or experimentally using a calibration table prepared in advance from prior experiments. For example, the above Equation (2) can be used for calibrating the g2(τ) itself for each measurement in real time, or the tabulated values from a calibration table obtained in advance (e.g., values as shown in FIG. 3A) can be used to calibrate certain parameters of g2(τ), as further explained below.

The display 220 in the operating console 201 of the DWS apparatus 200 can show the g2(τ) function, which is calibrated by using the actual source-detector distance, and related parameters, such as β values and time constant (τ) values extracted from the g2(τ) function, and also the measured real source-detector distance.

### Second Embodiment

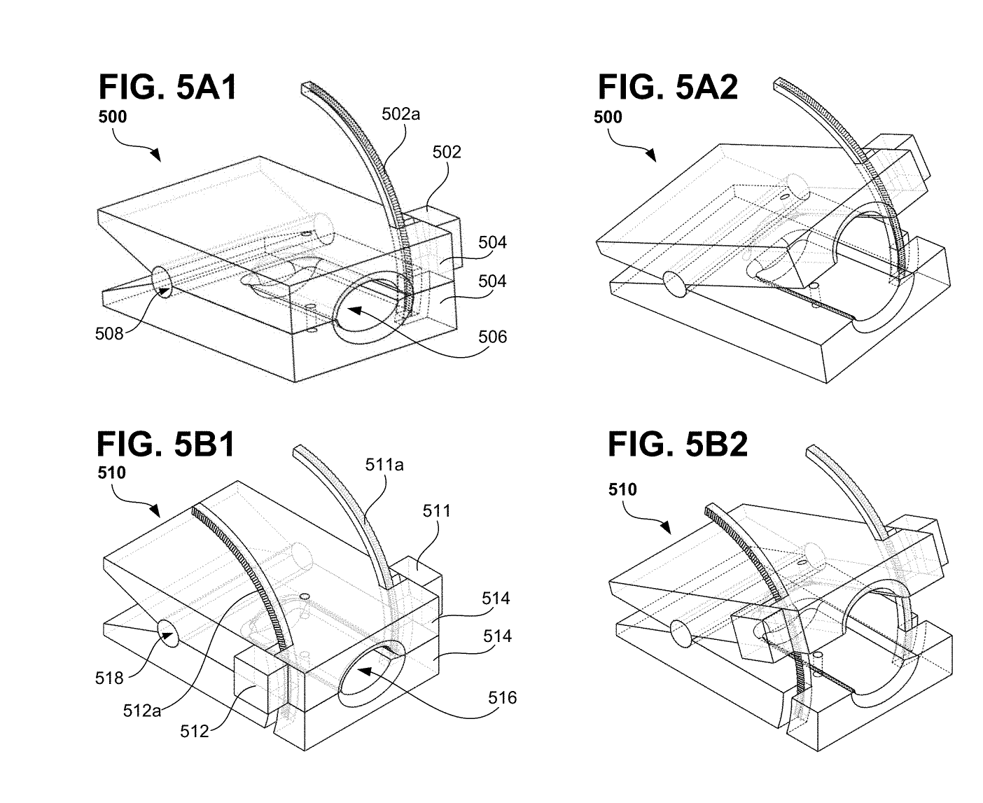

FIGS. 5A1-5A2 and 5B1-5B2 show examples of a DWS probe having integrated therein one or more linear encoders. FIG. 5A1 shows a probe 500 in the form of a finger clamp (clamping unit) in a closed state in which a linear encoder 502 has been integrated into the probe. FIG. 5A2 shows the probe 500 with a single encoder in an open state. FIG. 5B1 shows a probe 510 also in the form of a finger clamp (clamping unit) in an open state in which a first linear encoder 511 and a second linear encoder 512 have been integrated into the probe. FIG. 5B2 shows the probe 510 with multiple encoders in an open state.

In FIG. 5A1, the probe 500 includes a pair of clamp plates 504, a hinge portion 508, and a linear encoder 502 with a curved scale 502a. The probe 500 is similar to the above described probe 290 in that probe 500 includes a pair of clamp plates 504 that are mechanically joined by the hinge portion 508. The clamp plates 504 each include a notched section to form a sample receiving space 506 of a tubular shape having a circular cross-section. In this manner, the clamp plates 504 are configured to be opened and closed to receive therein a finger or thumb or toe of a subject (anatomical extremity) for examination. FIG. 5A1 shows that a stationary portion of the linear encoder 502 can be mechanically attached to one of the clamp plates 504 and the encoded strip (scale 502a) passes through both of the clamp plates 504. In this manner, every time either one the clamp plates 504 opens or closes, or moves relative to each other, and any time a sample moves or changes its position, the linear encoder 502 can detect a variation in the source-detector distance.

In FIG. 5B1, the probe 510 includes a pair of clamp plates 514, a hinge portion 518, a first encoder 511 and a second encoder 512. It is noted that encoder 511 and encoder 512 each includes a curved or bent encoded strip (511a and 512a, respectively). However, the operation of these encoders 511 and 512 is based on the above-described principle of a linear encoder. The probe 510 is similar to the above described probe 290 in that the probe 510 includes a pair of clamp plates 514 that are mechanically joined by the hinge portion 518. The clamp plates 514 each include a notched section to form a sample receiving space 516. In this manner, the clamp plates 514 are configured to be opened and closed to receive therein a finger or a thumb (an anatomical extremity) of a subject for examination.

FIG. 5B1 shows that a portion of each encoder 511 and 512 is mechanically attached to the upper one of the clamp plates 514 and the ends of the encoded strips (511a and 512a) of each encoder is mechanically attached to the lower one of the clamp plates 514. Both encoded strips pass through the upper one of clamp plates 514. In this manner, every time either one of the clamp plates 514 opens or closes, or one clamp plate moves relative to the other, and any time a sample moves or changes its position, the encoders 511 and 512 can detect a variation in the source-detector distance.

In FIGS. 5B1 and 5B2, stripe scales 511a and 512a are stripe lines and they are consisted of optically clear/opaque regions or uneven steps. The encoder 511 and encoder 512 are linear encoders and they are consisted of light sources and detectors. The stripe lines are attached to a clamp plate 514 so that the stripe lines can be moved under the linear encoders 511 and 512. When the stripe line with optically clear/opaque regions moves under the linear encoder, of the liner encoder moves with respect to the encoder stripe line, intensity of reflected light from the stripe line, which is detected at a detector, changes. When the stripe line with uneven steps moves under the linear encoder, positions of reflected light from the stripe line, which is detected at a detector, changes. Since the space of optically clear/opaque regions or uneven steps can be controlled, the linear encoder can measure the change of distance by counting the number of changes caused by the encoded stripes. In FIGS. 5B1 and 5B2, one advantage of having two encoders is that the first encoder can be used for coarse resolution measurement, while the second encoder can be used for fine resolution measurements. Alternatively, the second encoder can be used for redundancy. Alternatively or in addition, the first encoder can be used to measure the actual source-detector distance, and the second encoder can be used to monitor sample movement. Moreover, the second encoder can trigger a warning when sample movement is determined to be higher than a predetermined tolerance.

The main difference between the first embodiment and this second embodiment is the use of a linear encoder (LE) instead of using a rotary encoder. Since a typical linear encoder size is less than 10 mm with ˜100 μm resolution, a linear encoder can be incorporated into a clamping unit at any position. The position of one or more linear encoder(s) is shown in FIGS. 5A1, 5A2, 5B1 and 5B2, but the type and number of linear encoders and the positioning thereof is not limited to the disclosed embodiment(s).

With respect to the linear encoders shown in probes 500 and 510, the source-detector distance can be measured in a manner similar to that described above with respect to the rotary encoder of FIGS. 4A-4B. Specifically, as illustrated in FIGS. 5A1-5B2, the encoded scales (stripes) 502a, 511a and 512a are not strictly linear but arcuate or curved. Therefore, the source-detector distance is defined by an arc length similar to the above-described case of rotary encoders. One difference with respect to the rotary encoder is that the distance resolution in linear encoders is defined by the number of pulses per unit distance (pulse per millimeter or pulse per inch) instead of pulses per revolution. Therefore, the distance resolution of linear encoders can be much higher than that of rotary encoders.

### <Control System and Control Process for DWS Apparatus>

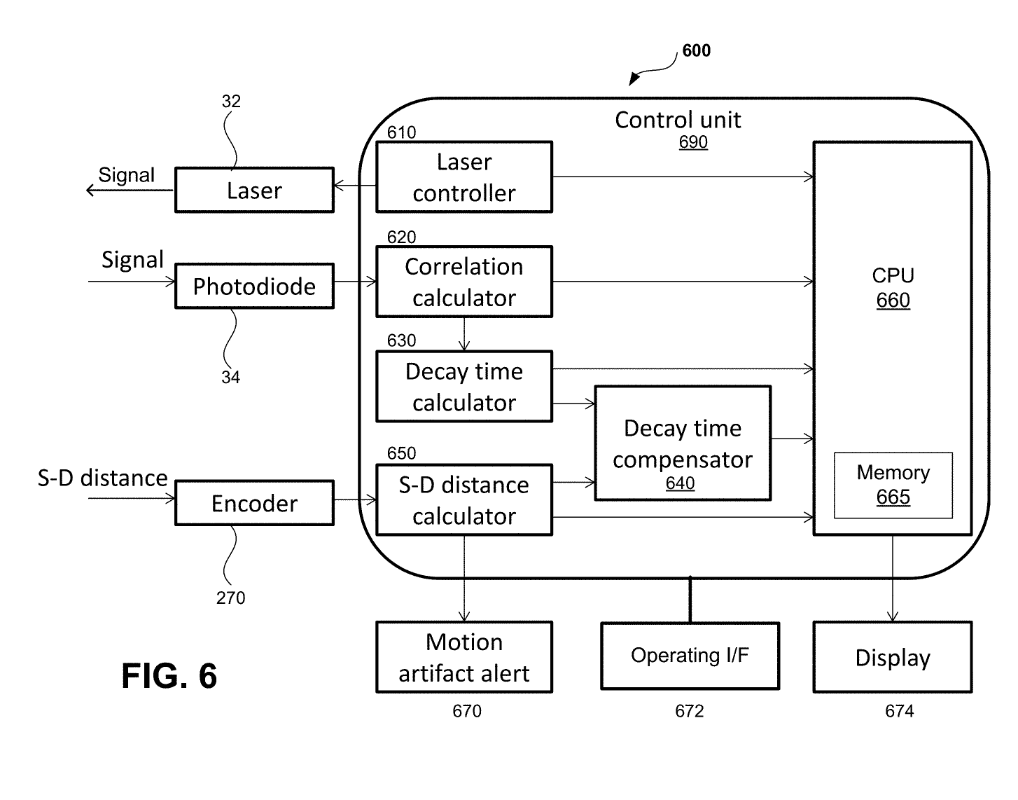

FIG. 6 illustrates a schematic block diagram of a control system for performing DWS measurements and calibrating the autocorrelation function by using the source-detector distance information. As illustrated in FIG. 6, a control system 600 includes a control unit 690 which is operatively connected to a light source 32, a photodetector 34, and an encoder 270. The control unit 690 is also connected to input and output devices such as a motion artifact (alert output unit 670), a user operating interface (operating unit 672), and a display (display unit 674).

The control unit 690 is the equivalent of computer 210 illustrated in FIG. 2. The control unit 690 includes a laser controller 610, a correlation calculator 620, a decay time calculator 630, a decay time compensator 640, a source-detector (S-D) distance calculator 650, which are all operatively connected to a central processing unit (CPU) 660 and memory 665. The central processing unit (CPU) 660 represents one or more processors and performs overall control functions for the control system 600. The CPU uses a random access memory (RAM) included in memory 665 as a work area while executing instructions exemplified by the FIGS. 6-9. The CPU executes instructions of various programs stored in one or more memory devices. For example, the CPU executes programs stored in a read only memory (ROM) and in a storage device collectively illustrated as memory 665.

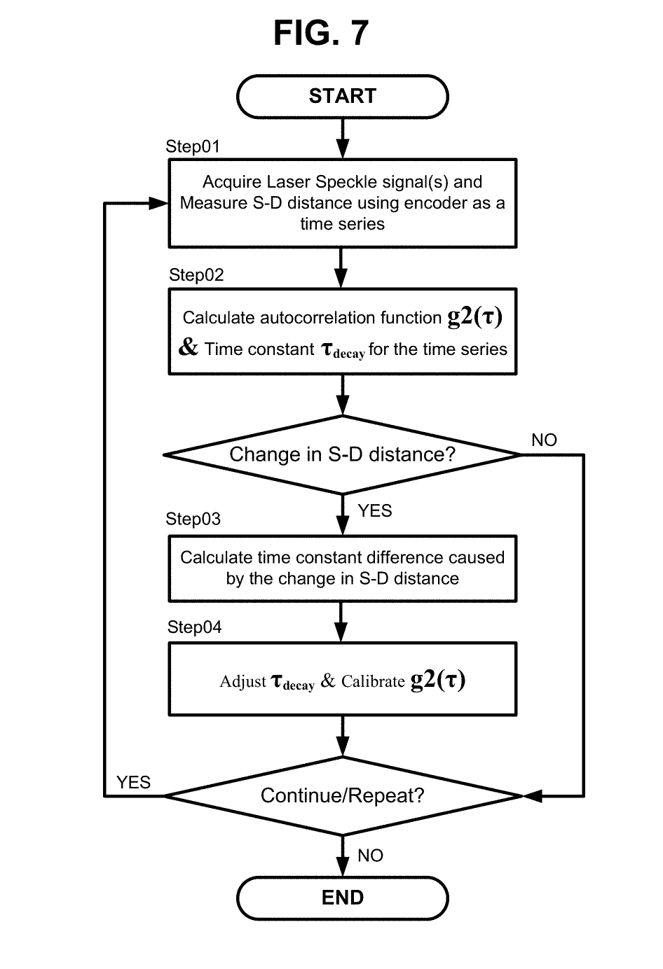

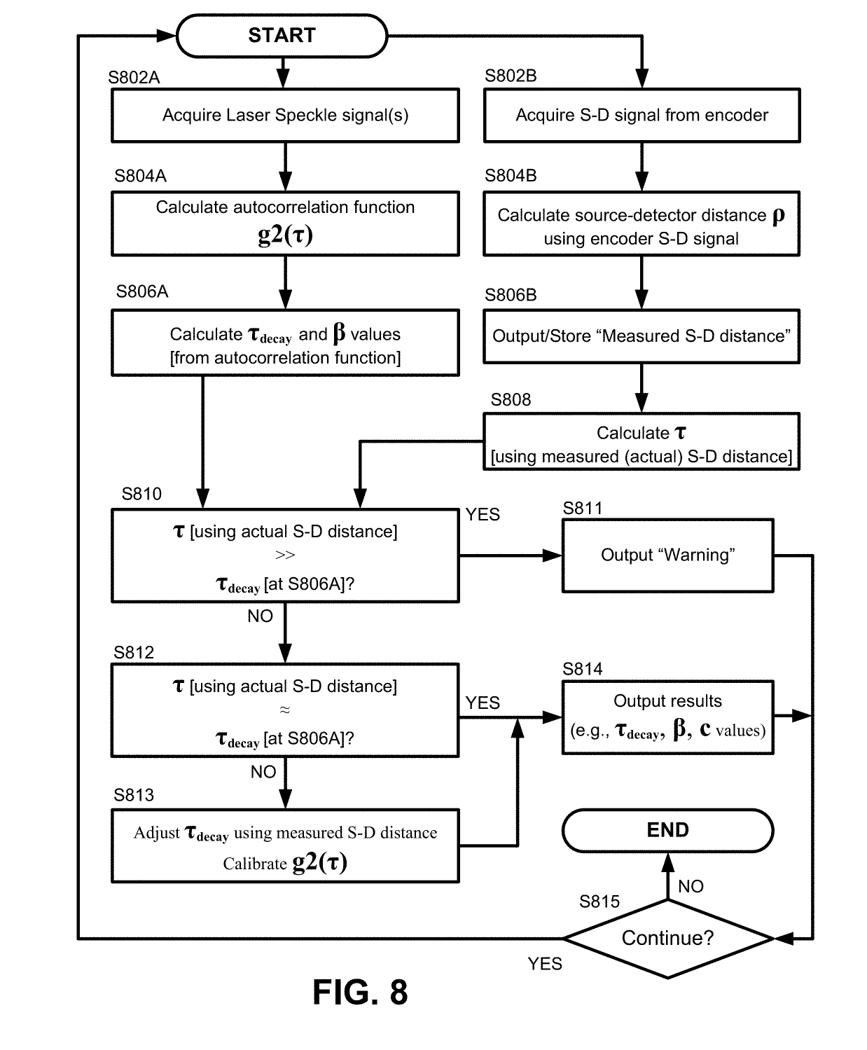

The CPU 660 is configured to read and execute computer-executable instructions stored in the memory 665. The computer-executable instructions include instructions for the performing the methods and/or calculations described herein. For example, in performing DWS measurements, the CPU 660 reads computer-executable instructions from memory 665 to calculate speckle fluctuations, as temporal intensity fluctuations, of light diffused through the sample. Or, CPU 660 reads computer-executable instructions from memory 665 to calculate the source-detector distance using data obtained from encoder 270. Furthermore, the CPU 660 reads computer-executable instructions from memory 665 to calculate a calibration value to calibrate one or more parameters of the autocorrelation function, by using the source-detector distance data. The operations of CPU 660 are described more in detail below by referring to the flow diagrams of FIG. 7 and FIG. 8.

FIG. 7 illustrates a flow diagram of a process implemented to control a DWS apparatus to perform DWS measurements, and to calibrate the intensity autocorrelation function by adjusting the time constant (time decay) of the autocorrelation function based on the actual source-detector distance measured by the encoder. According to FIG. 7, the flow process starts (START) when the DWS apparatus is in an operational state, for example, when the DWS probe receives therein a sample (e.g., tissue of a subject). In this operational state of the DWS apparatus 200, for example, at Step01, the encoder 270 outputs a S-D signal which is transmitted to the computer 210 (control unit 690 of FIG. 6). At the same time, at Step01, the source-detector pair (252 254) generates and outputs a DWS signal which is also received at the computer 210 (correlation calculator 620 of control unit 690 in FIG. 6). At step02, the correlation calculator 620 calculates the autocorrelation function g2(τ). At the same, at step02, the correlation calculator 620 derives the time decay (τ_{decay}) for all measurements in a time series manner.

To calculate an autocorrelation function g2(τ) from the DWS signal in the correlation calculator 620, the following Equation (3) is used.

$${g}_{2} ( τ ) = \frac{〈 I ( t ) I ( t + τ ) 〉}{{〈 I ( t ) 〉}^{2}} \qquad Equation ( 3 )$$

In Equation (3), I(t) is the DWS intensity signal at time t, τ is correlation time lag (time between consecutive signals in the time series), and the angular brackets < > denote time averaging.

The decay time calculator 630 derives a correlation time decay (τ_{decay}) value using the following fitting Equation (4).

$${{g}_{2} ( τ )}_{fit} = 1 + β \cdot {e}^{- \frac{τ}{{τ}_{decay}}} \qquad Equation ( 4 )$$

where β is the coherence factor, τ_{decay} is the time decay or time constant of the autocorrelation function, and τ is correlation time lag, as explained above.

At step02, the S-D distance calculator 650 determines in a change in S-D distance has occurred in the time series signals. If no change in S-D distance has occurred, any change in time decay values can be assumed to have been caused by tissue or blood conditions of the subject, and no calibration is necessary. On the other hand, if at step02, the S-D distance calculator 650 determines that a change in S-D distance has occurred in the time series signals, calibration of the time constant is necessary and the flow proceeds to step03.

At step03, if the change in S-D distance is excessive (higher than a threshold), the S-D distance calculator 650 may issue a motion artifact alert 670 (warning). In step03, the CPU 660 used the change in S-D distance to calculate a time constant difference caused by the change in S-D distance.

At step04, the decay time compensator 640 (calibration unit) uses the measured source-detector signal (source-detector information data) to adjust (calibrate) time decays of the autocorrelation function(s) g2(τ) using a calibration equation, for example, Equation (2). The process is iteratively repeated until all necessary measurements are performed or an active “Stop” command is input by the user at step04.

The decay time compensator 640 works under the following principles. The decay time compensator 640 uses a calibration equation of the form y=f(x), which is established based on prior experiments or previously known values. For example, the equation of calibration can be based on values of Equation (1) or Equation (2). The calibration process can be performed as the following operation of Equation (5).

yₜ_{_{2—}}_{calibrated}=yₜ_{₁}−{f(xₜ_{₁})−f(xₜ_{₂})} Equation (5)

Where x is the S-D distance, y is a value of time decay function, t₁ is a time after t₁ seconds have passed from a start time t₀, and t₂ is a time after a minimum time increment has passed from t₁. When the time decay (τ_{decay}) is completely the same at t=t₁ and t=t₂ but only the S-D distance is different, yₜ_{_{2—}}_{calibrated} is calculated, for example using Equation (5). On the other hand, when the time decay (τ_{decay}) at t=t₁ is different from that at t=t₂, but the S-D distance has not changed, this difference is caused by a change in tissue or blood condition, yₜ_{_{2—}}_{calibrated} is not same as yₜ_{₂}, but this difference does not include the effect of S-D distance change. Therefore, when there is no change in S-D distance, a change in time decay (τ_{decay}) can be safely attributed a change in tissue or blood condition.

A brief summary of a calibration flow process is as follows. First, Step01, measure speckle intensity (e.g., use Equation 3), and measure the S-D distance (Xₜ₀, Xₜ₁, Xₜ₂, . . . ) by an encoder as a time series (t0, t1, t2, . . . tn). Second, Step02, calculate time constant (τ_{decay}_{_}ₜ₀, τ_{decay}_{_}ₜ₁, τ_{decay}_{_}ₜ₂, . . . τ_{decay}_{_}ₜₙ) using Equation (4) as a time series. Third, calculate a calibration value (f(Xₜ₀), f(Xₜ₁), f(Xₜ₂), . . . f(Xₜₙ)) by a predetermined equation f(xₜ). In one example, as explained above, f(x)=0.0059e^{-0.162x}^{ₜ}. Fourth, step03, calculate time constant difference caused by the change in S-D distance, e.g., every successive measurement (f(Xₜ₁)−f(Xₜ₂)). Fifth, Step04, if time constant difference is caused by a change in S-D distance, perform calibration (τ_{decay}_{_}ₜ₂_{_}_{calibrated}=τ_{decay}_{_}ₜ₂−{f(xₜ₁)−f(xₜ₂)}.

More specifically, in operation, in response to a user input, the laser controller 610 activates laser 32 to emit a coherent light signal. The light is delivered via a first single mode fiber (SMF) to the sample (e.g., an anatomical extremity), the light diffuses through the sample, and at least a part of the diffused light is collected and then delivered by a second SMF to the photodetector 34, as described above in reference to FIGS. 2A-2B. At the same time, the encoder 270 constantly monitors the source-detector distance. The control unit 290 receives a first signal output from the photodetector 34 and a second signal output from the encoder 270. For purposes of distinction, the first signal output from the photodetector 34 will be referred to as a DWS signal, while the second signal output from the encoder 270 will be referred to as an S-D signal (or distance signal).

The correlation calculator 620 uses the DWS signal to calculate an autocorrelation function g2(τ), as described above. From the correlation function g2(τ), the decay time calculator 630 derives a time decay (τ_{decay}) value. Similarly, the S-D distance calculator 650 (distance measuring unit) uses the S-D distance signal output by the encoder 270 to calculate the actual physical distance between the source terminal (source 252) and the detector terminal (detector 254) to obtain an source-detector (S-D) distance (ρ) value. The decay time compensator 640 receives both the measured S-D distance value and the calculated time decay value and compares the two values using calculated source-detector distance data, for example, using data from FIG. 3A.

In this manner, the decay time compensator 640 compares the actual S-D distance value (ρ) to the value derived from the time decay (τ_{decay}), and provides at least one of the following results. First, if the two values are within a certain tolerance value, e.g., within 1% to 5% from each other, the autocorrelation function g2(τ) or any value derived therefrom can be verified as accurate and valid. Second, if the two values are not equal or within 1% to 5% from each other, the autocorrelation function g2(τ) can be compensated adjusted using a calibration. Third, if after a attempting to correct the correlation function, the two values (the actual S-D distance and the time decay) cannot be converged, it is possible to add a motion artifact alert, which warns a user when a change of S-D distance value (ρ) goes over a predetermined threshold value.

Here, it should be noted that because the value derived from the time decay (τ_{decay}) can be affected by the change of tissue or blood conditions, the “threshold” for a motion artifact alert can work only by using the actual S-D distance. Then, the implementation of a motion artifact alert is envisioned in the following manner.

A motion artifact alert warns a user when a change of actual S-D distance goes over a predetermined threshold. The purpose of this alert is to tell a user that the sample under examination (finger or body part) may be moving or has changed its position. To that end, for example, the threshold can be defined as the following.

$$\frac{{ρ}_{{t}_{2}} - {ρ}_{{t}_{1}}}{{ρ}_{{t}_{1}}} \geq 10 \%$$

That is, if the change of actual S-D distance ρ from t₁ to t₂ compared to p at t₁ becomes over (higher than) 10%, the motion artifact alert warns a user. The threshold should not exceed 10% for the measurement to remain within a range that can be calibrated. More specifically, as discussed above, measurement results within a 4% of the expected value can be considered “good enough”, and calibration thereof can be omitted. For measurement results above a 10% of the expected value, a motion artifact is output, and calibration thereof may not be possible; therefore the measurement process may be restarted. For measurement results between 4 to 10% (higher than 4% and less than 10%) calibration can be performed as described herein below in reference to FIG. 8.

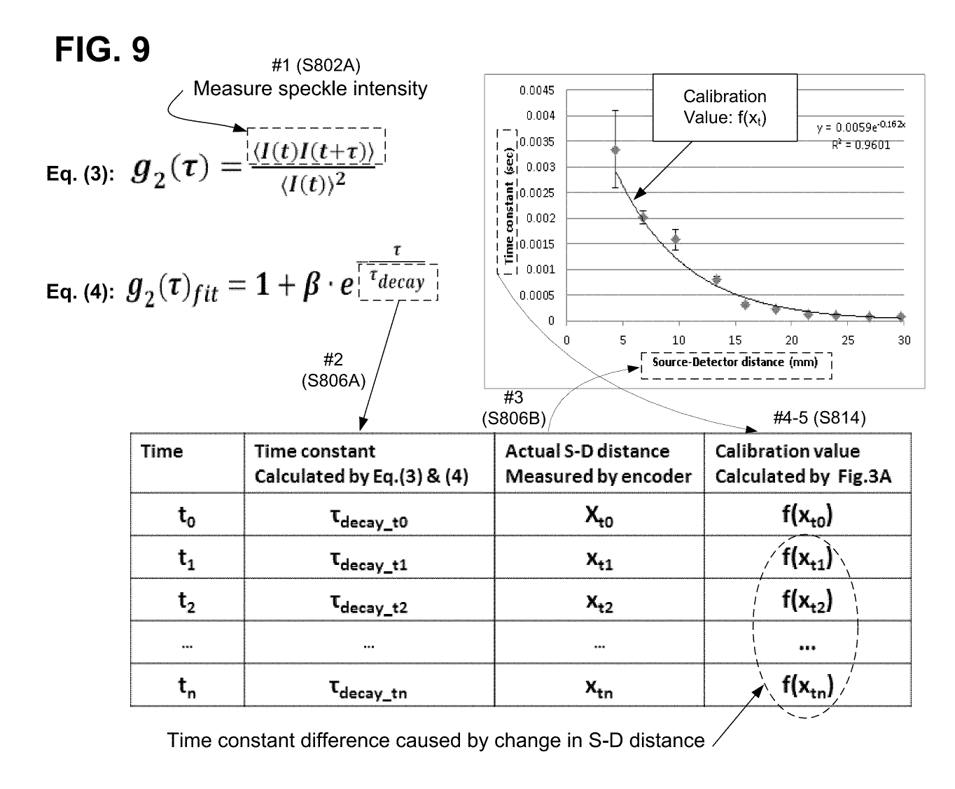

FIG. 8 illustrates another flow diagram of an algorithm implemented to control a DWS apparatus to perform DWS measurements, and to calibrate the intensity autocorrelation function by using the measured actual source-detector distance. FIG. 9 diagrammatically shows how the algorithm of FIG. 8 makes use of actual source-detector distance to adjust the intensity autocorrelation function.

As in the case of FIG. 7, in the process of FIG. 8 too, the flow assumes that the DWS system is in an operative state after an START signal. Therefore, in an operative state, at step S802A, the DWS system acquires one or more laser speckle signals using the above-described probe 290. Specifically, the laser controller 610 activates the laser 32 to emit light towards a sample or patient. In response thereto, the photodiode 34 detects light that has diffused through the sample, and outputs a DWS signal. At the same time, in step S802B, the encoder 270 continuously monitors the physical source-detector distance and outputs an S-D distance signal.

At step S804A, the correlation calculator 620 uses the DWS signal received from the photodiode 34, and calculates a correlation function g2(τ). Similarly, at step S804B, the S-D distance calculator 650 uses the S-D distance signal from the encoder 270 to calculate the actual distance between the source 252 and the detector 254 (see, e.g., FIG. 2B). Here, as explained above, the output from encoder 270 is used to calculate the actual S-D distance which accounts for any sample movement or probe misplacement due to, for example, the sample's anatomy or patient movement.

Regarding the output of the encoder, there are two types output, absolute and relative values. When the clamp plates are closed, a user or the control unit can set the S-D distance as zero or an initial value, which is mechanically determined S-D distance. In this case, the output of encoder is absolute values. On the other hand, as discussed above, it is important to monitor the change in S-D distance during measurement. However, because of movement, it may be difficult to output absolute values. Then, even if the encoder only outputs relative values indicating a change in the absolute values, it is still necessary to calibrate the time decay due to sample movement. Therefore, the output of encoder can be absolute and/or relative values.

At step S806A, the decay time calculator 630 uses the correlation function g2(τ) to calculate or derive characteristic parameters of the correlation function, such as the time decay (τ_{decay}) and β values. Here, the decay time calculator 630 calculates the time decay (τ_{decay}) taking into consideration the general parameters of the sample, e.g., tissue conditions, blood flow levels, etc. That is, at step S806A, the time decay (τ_{decay}) values are calculated as in a conventional manner without taking into consideration the signal S-D distance measured by the encoder.

In addition, at S806A, when the fitting equation is modified as the following Equation (6),

$${{g}_{2} ( τ )}_{fit} = c + β \cdot {e}^{- \frac{τ}{{τ}_{decay}}} \qquad Equation ( 6 )$$

where β is the coherence factor, τ_{decay} is the time decay of the correlation function, also known as a time constant, and c is a plateau level. Then the decay time calculator 630 can also derive the plateau level c of the intensity correlation function.

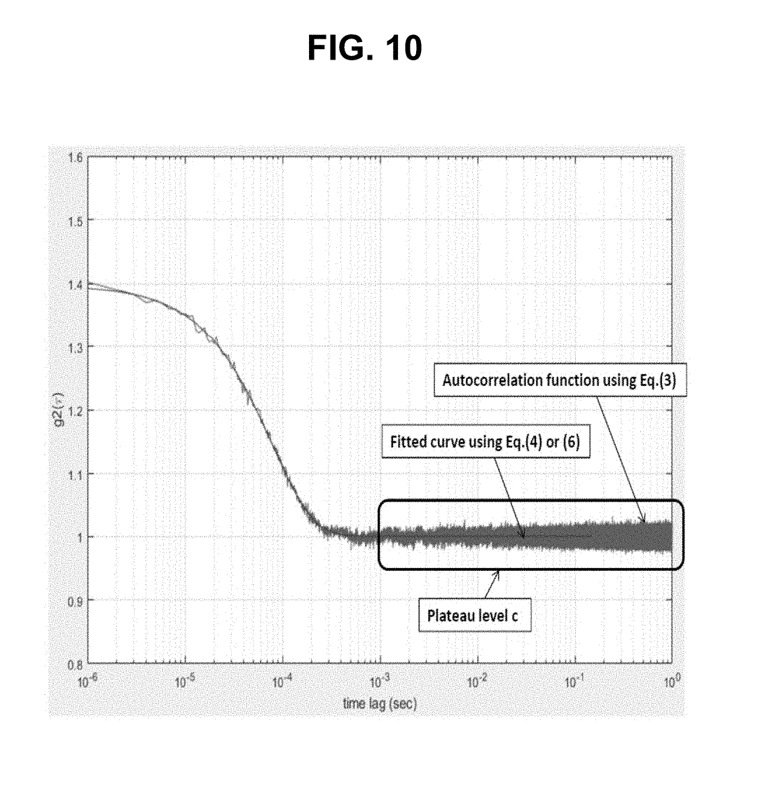

FIG. 10 shows an example of g2(τ) with the plateau level c. The plateau level c of normalized g2(τ) should be 1, then Equation (4) is enough for fitting. But in some cases, certain factors such as patient motion, electric noise, periodic mechanical vibration, and the like can cause the plateau level c to be substantially different from 1. In those situations, using Equation (6) can avoid the selection (fitting) of deteriorated and ineffective g2(τ) values, because the plateau level is far away from 1.

Referring back to FIG. 8, at step S806B, the S-D distance calculator 650 stores in memory 665 the measured (actual) source-detector distance. Typically the measured S-D distance can be stored and updated for every measurement cycle, or can averaged every certain number of cycles of measurements.

At step S808, the decay time calculator 630 uses the actual (measured) S-D distance to calculate the actual time constant (τ) of the correlation function g2(τ). That is, at step S808, the decay time calculator 630 determines the correlation time constant (τ) using the actual “S-D distance”. To obtain the time constant based on the S-D distance, data from DWS measurement results, for example, a table of values for the graph as that illustrated in FIG. 3 can be useful. Alternatively, at step S808, Equation (2) is used.

At step S810, the decay time compensator 640 receives the value of the time constant (τ) based on actual S-D distance measured by the encoder (obtained at step S806B), and the calculated time decay value (τ_{decay}) obtained at step S806A. And based on this information, the decay time compensator 640 compares the time constant (τ) of the actual measured S-D distance against the time decay (τ_{decay}) calculated at S806A without the actual S-D distance. If the time decay calculated at S806A is within threshold value of the time constant (τ) based on the measured S-D distance (e.g., if difference between τ_{decay} and τ calculated using the actual S-D distance is less than 10%), the process proceeds to step S811. On the other hand, if the difference is above the threshold value (e.g., if the difference between τ_{decay} and τ calculated using the actual S-D distance is equal to or greater than 10%), the decay time compensator 640 determines that a change in the S-D distance during the DWS measurement has occurred, and that such S-D distance change is over a predetermined threshold value. In this case, the process proceeds to step S811.

At step S811, the CPU 660 issues a motion artifact alert (a “warning”) via the alert output unit 670 (e.g., the CPU 660 outputs a beeping sound or vibration alert, or displays such warning via display 674). Then the process advances to step S815. That is, if the decay time compensator 640 determines that a change in S-D distance during measurement goes over a predetermined threshold such that calibration is not possible, the CPU 660 warns the user. At this point, at step S815, the user may choose to stop or continue the DWS measurements. For example, the user can stop and reposition the probe on the patient or sample before restarting the measurement. In other words, at step S815, the calibrating process may include outputting a warning and rearranging the probe on the patient.

At step S812, since it has been already determined at step S810 that the time decay value τ_{decay} and the time constant τ calculated using the actual S-D distance are within a certain tolerance (below a threshold), the decay time compensator 640 now determines whether that certain tolerance is within a negligible margin or not (e.g., determines if the difference is not greater than 4%). Depending on the desired degree of accuracy of the measurement (depending on a desired degree of similarity between the time decay τ_{decay} value without consideration for the actual S-D distance and the time constant τ calculated using the actual S-D distance), the decay time compensator 640 determines whether or not to calibrate the correlation function.

Specifically, at step S812, if the time decay τ_{decay} value calculated at S806A and the time constant τ value calculated at S808 based on the actual S-D distance are greater than a negligible difference (e.g., a difference greater than 4%) and lower than a motion artifact threshold (lower than 10%), the process proceeds to S813 where the decay time compensator 640 adjusts the time decay (τ_{decay}) value using the measured actual S-D distance and using a calibration equation (e.g., Equation (2) y=0.0059ê(−0.162x), where y is the value of the time constant (τ) and x is the measured actual source-detector distance. In this manner, the correlation function can be adjusted (calibrated) by taking into account the actual S-D distance measured by the distance measurement unit (encoder). From step S813, the process advances to S814.

At step S812, if the time decay (τ_{decay}) value calculated at S806A and the time constant value (τ) calculated based on the actual S-D distance (calculated at S806B) are approximately equal to each other (about the same), the process proceeds to step S814 directly, without calibrating the correlation function. At step S814, the CPU 660 outputs the measurement results, such as τ_{decay} and β values, via the display 674. Naturally, these accurate results can also be stored in memory 665. Again, at this point, the flow process advances to step S815, where the user may chose to stop or continue the DWS measurements. As used herein, the term “about,” means between 0 and 10% of the value or more preferably between 0 and 5% of the value.

Here, it should be understood that in the flow of steps S810, S812, S813 to S814 of FIG. 8, the decay time compensator 640 calculates a calibration value (f(Xₜ₀), f(Xₜ₁), f(Xₜ₂), . . . f(Xₜₙ)) according to the time series, by the predetermined equation f(xₜ), and calculates a time constant difference caused by a change of S-D distance (f(Xₜ₁)−f(Xₜ₂)) to perform the calibration of the time decay value (τ_{decay}), as follows: (τ_{decay}_{_}ₜ₂_{_}_{calibrated}=τ_{decay}_{_}ₜ₂−{(f(xₜ₁)−f(xₜ₂)}. That is, as explained above, when the time decay (τ_{decay}) calculated at S806A is completely the same at t=t₁ and t=t₂ but only the S-D distance is different, the decay time compensator 640 calculates a calibration value using a calibration Equation which takes into consideration the S-D distance change. On the other hand, when the time decay (τ_{decay}) at t=t₁ is different from that at t=t₂, but the S-D distance has not changed, this difference is caused by a change in tissue or blood condition, but this difference does not include the effect of S-D distance change, the decay time compensator 640 does not need to calculate a calibration value using the change in S-D distance. Therefore, when there is no change in S-D distance, a change in time decay (τ_{decay}) can be safely attributed a change in tissue or blood condition.

The display 674 (in FIG. 6) can show laser parameters, and/or correlation curves, and/or decay times, and/or S-D distances during measurement. More specifically, the display 674 can be configured to show interactively the DWS system operation, so that a user can confirm that the calculated autocorrelation function g2(τ) is based on accurate source-detector distance data.

Therefore, as described above, it is advantageous to exclude the influence of change in the source-detector distance due to sample movement or patient anatomy from the measurement of time decay of the intensity autocorrelation function curve. The properties such as the flow of the moving particles in the sample are estimated from the time constant of autocorrelation function measured by a DWS system. Since DWS measurements are highly dependent on, and sensitive to, the source-detector distance, it is important monitor and confirm that an accurate source-detector distance is used in such measurements. The time constant or time decay of the autocorrelation function measured using DWS system is changed by a change the source-detector distance. When the patient moves, the source-detector distance and the time constant may be changed. In the conventional DWS system, it is difficult to identify the reason why the time constant was changed. However, by employing the scheme as disclosed herein which measures the actual source-detector distance, it is possible to identify the reason why the time constant changes, and thus it is possible to make adjustments to obtain more accurate results.

While the present patent application has been described with reference to exemplary embodiments, it is to be understood that the invention is not limited to the disclosed exemplary embodiments. The scope of the following claims is to be accorded the broadest interpretation so as to encompass all possible modifications and equivalent structures and functions. To that end, it must be noted that the terminology used herein is for the purpose of describing particular embodiments only and is not intended to be limiting.

As used herein, the singular forms “a”, “an”, and “the”, are intended to include the plural forms as well, unless the context clearly indicates otherwise. It should be further understood that the terms “includes” and/or “including”, when used in the present specification and claims, specify the presence of stated features, integers, steps, operations, elements, and/or components, but do not preclude the presence or addition of one or more other features, integers, steps, operations, elements, components, and/or groups thereof not explicitly stated.

What is claimed is:

1. A diffusing wave spectroscopy apparatus, comprising:
a source configured to emit light to irradiate tissue of a subject;
a detector configured to detect at least part of the light after propagation through the tissue;
a distance measurement unit configured to measure a source-detector distance;
a control unit configured to obtain an intensity autocorrelation function by using light intensity data corresponding to light intensity signals obtained from the detector, and to derive a time decay value (τ_{decay}) of the intensity autocorrelation function; and
a calibrating unit configured to calibrate the intensity autocorrelation function by adjusting the time decay value of the intensity autocorrelation function based on the measured source-detector distance.

2. The diffusing wave spectroscopy apparatus according to claim 1, wherein
the source includes a coherent light source and a source optical fiber, the source optical fiber having a proximal end thereof connected to the coherent light source and a distal end thereof arranged in contact with the tissue of the subject, and
the detector includes a photodetector and a detector optical fiber, the detector optical fiber having a proximal end thereof connected to the photodetector and a distal end thereof arranged in contact with the subject at a location other than the distal end of the source optical fiber.

3. The diffusing wave spectroscopy apparatus according to claim 2, wherein the distance measurement unit measures a distance between the distal end of the source optical fiber and the distal end of the detector optical fiber both in contact with the subject, as the source-detector distance.

4. The diffusing wave spectroscopy apparatus according to claim 1, wherein the distance measurement unit includes at least one linear encoder.

5. The diffusing wave spectroscopy apparatus according to claim 1, wherein the distance measurement unit includes at least one rotary encoder.

6. The diffusing wave spectroscopy apparatus according to claim 1, wherein the probe includes a clamp configured to receive therein an anatomical extremity of a subject.

7. The diffusing wave spectroscopy apparatus according to claim 1,
wherein the calibrating unit is configured to calibrate the intensity autocorrelation function by adjusting the time decay value (τ_{decay}) of the intensity autocorrelation function according to the following equation y=0.0059ê(−0.162x),
where y is a value of a time decay function and x is the measured source-detector distance.

8. The diffusing wave spectroscopy apparatus according to claim 1,
wherein the calculation unit derives a time decay value (τ_{decay}) of the intensity autocorrelation function based on tissue or blood condition of the subject,
wherein the calculation unit is further configured to calculate a time constant value (τ) of intensity autocorrelation function based on the measured source-detector distance, and
wherein the calibration unit is configured to calibrate the intensity autocorrelation function by comparing the time decay value (τ_{decay}) of the intensity autocorrelation function to the time constant value (τ) calculated using the measured source-detector distance, and determining whether a difference thereof is below a threshold value.

9. The diffusing wave spectroscopy apparatus according to claim 7, wherein the calibration unit calibrates the intensity autocorrelation function when the difference is below the threshold value.

10. The diffusing wave spectroscopy apparatus according to claim 7, wherein the calibration unit is configured to output a warning signal when the difference is equal to or above the threshold value.

11. A method of calibrating measurements of a diffusing wave spectroscopy apparatus, comprising:
emitting a coherent light from a source to irradiate tissue of a subject;
detecting, using a detector, light intensity of scattered light after propagation of the light through the tissue of the subject;
measuring a source-detector distance;
calculating an intensity autocorrelation function by using light intensity data corresponding to light intensity signals obtained from the detector, and deriving a time decay value of the intensity autocorrelation function; and
calibrating the intensity autocorrelation function by adjusting the time decay value of the intensity autocorrelation function based on the measured source-detector distance.

12. The method according to claim 11, wherein measuring the source-detector distance includes measuring a distance between a distal end of a source optical fiber and a distal end of a detector optical fiber both in contact with the subject and separate from each other.

13. The method according to claim 12, wherein measuring the source-detector distance includes measuring a distance between the distal end of the source optical fiber and the distal end of the detector optical fiber using at least one linear encoder.

14. The method according to claim 12, wherein measuring the source-detector distance includes measuring a distance between the distal end of the source optical fiber and the distal end of the detector optical fiber using at least one rotary encoder.

15. The method according to claim 11,
wherein calibrating the intensity autocorrelation function includes adjusting the time decay value (τ_{decay}) of the intensity autocorrelation function according to the following equation y=0.0059ê(−0.162x),
where y is a value of a time decay function and x is the measured source-detector distance.

16. The method according to claim 11, further comprising:
calculating a time constant value (τ) of the intensity autocorrelation function based on the measured source-detector distance,
wherein the deriving a time decay value (τ_{decay}) of the intensity autocorrelation function is based on tissue or blood condition of the subject, and
wherein calibrating the intensity autocorrelation function includes comparing the time decay value (τ_{decay}) of the intensity autocorrelation function to the time constant value (τ) calculated using the measured source-detector distance, and determining whether a difference thereof is below a threshold value.

17. The method according to claim 16, wherein, when the difference is below the threshold value, calibrating the intensity autocorrelation function includes adjusting the time decay value (τ_{decay}) of the intensity autocorrelation function according to the following equation
yₜ_{_{2—}}_{calibrated}=yₜ_{₁}−{f(xₜ_{₁})−f(xₜ_{₂})}
where x is the measured source-detector distance, y is a value of a time decay function, t₁ is a time after t₁ seconds have passed from a start time t₀, and t₂ is a time after a minimum time increment has passed from t₁.

18. The method according to claim 16, wherein, when the difference is equal to or above the threshold value, calibrating the intensity autocorrelation function includes outputting a warning signal and rearranging the source and detector on the subject.

19. A system comprising:
one or more processors; and
one or more computer-readable media coupled to the one or more processors, the one or more computer-readable media storing instructions that, when executed by the one or more processors, cause the one or more processors to perform operations comprising:
emitting a coherent light from a source to irradiate tissue of a subject;
detecting, using a detector, light intensity of scattered light after propagation of the light through the tissue of the subject;
measuring a source-detector distance;
calculating an intensity autocorrelation function by using light intensity data corresponding to light intensity signals obtained from the detector, and deriving a time decay value of the intensity autocorrelation function; and
calibrating the intensity autocorrelation function by adjusting the time decay value of the intensity autocorrelation function based on the measured source-detector distance.

20. A computer-readable medium storing instructions that, when executed by one or more processors, cause the one or more processors to perform operations comprising:
emitting a coherent light from a source to irradiate tissue of a subject;
detecting, using a detector, light intensity of scattered light after propagation of the light through the tissue of the subject;
measuring a source-detector distance;
calculating an intensity autocorrelation function by using light intensity data corresponding to light intensity signals obtained from the detector, and deriving a time decay value of the intensity autocorrelation function; and
calibrating the intensity autocorrelation function by adjusting the time decay value of the intensity autocorrelation function based on the measured source-detector distance.
